(19) United States
(12) Patent Application Publication
(10) Pub. No.: US 2007/0104648 A1
(43) Pub. Date: May 10, 2007

(54) Compositions, methods of preparing amino acids, and nuclear magnetic resonance spectroscopy

(75) Inventor: Brian Keith Shull, Durham, NC (US)

Correspondence Address: Peter G. Carroll;MEDLEN & CARROLL, LLP, Suite 350, 101 Howard Street, San Francisco, CA, 94105, US

(73) Assignee: Glyconix Corporation

(21) Appl. No.: 11/594,299

(22) Filed: Nov. 8, 2006

Related U.S. Application Data
(60) Provisional application No. 60/734,815, filed on Nov. 9, 2005.

(51) Int. Cl. A61K 49/00

(52) U.S. Cl. 424/9.3

(57) ABSTRACT

The present invention relates to amino acids, complexes, and compounds comprising deuterium and tritium isotopes preferably alpha deuterated amino acids, polypeptides, antibodies, derivatives and saccharide-amino acid complexes and conjugates. In some embodiments, the invention relates to methods of using compounds comprising deuterium for imaging biochemical concentrations and distributions in mammalian tissues using nuclear magnetic resonance spectroscopy. In some embodiments, the invention relates to the used of said amino acids derivatives and complexes in boron neutron capture therapy. In some embodiments, the present invention relates to the preparation of amino acids, polypeptides, antibodies, derivatives and saccharide complexes/conjugates comprising heavy hydrogen isotopes. In some embodiments, the invention relates to racemizing amino acids starting from compositions of any optical purity. In further embodiments, the invention relates to the preparation of amino acids and their N-acyl counterparts with deuterium incorporated at the alpha carbon.

### FIELD OF THE INVENTION

The present invention relates to amino acids, complexes, and compounds comprising deuterium and tritium isotopes preferably alpha deuterated amino acids, polypeptides, antibodies, derivatives and saccharide-amino acid complexes and conjugates. In some embodiments, the invention relates to methods of using compounds comprising deuterium for imaging biochemical concentrations and distributions in mammalian tissues using nuclear magnetic resonance spectroscopy. In some embodiments, the invention relates to the used of said amino acids derivatives and complexes in boron neutron capture therapy. In some embodiments, the present invention relates to the preparation of amino acids, polypeptides, antibodies, derivatives and saccharide complexes/conjugates comprising heavy hydrogen isotopes. In some embodiments, the invention relates to racemizing amino acids starting from compositions of any optical purity. In further embodiments, the invention relates to the preparation of amino acids and their N-acyl counterparts with deuterium incorporated at the alpha carbon.

### BACKGROUND OF THE INVENTION

Glioblastoma Multiforme (GBM) is the most common and most aggressive of the primary brain tumors. Boron neutron capture therapy is useful for the treatment of GBM. Currently, protocols that use ¹⁰B enriched para-boronophenylalanine (p-boronophenylalanine or p-BPA) complexes for the treatment of GBM call for the infusion of large quantities over the course of several hours, followed by subsequent exposure to isothermal neutrons. To maximize the radiation effect, the neutrons should be delivered when the ratio between the boron concentrations from the complexes in tumor cells to that in normal tissues reaches maximum. The pharmacokinetics of p-BPA and other boron delivery agents are only partly known. Monitoring p-BPA in a subject is generally accomplished by repeated central venous blood sampling followed by ex vivo assay of the ¹⁰B concentration by prompt-gamma neutron activation analysis. Although an accurate evaluation of the ¹⁰B concentration in the blood, it does not provide the concentration of ¹⁰B in the tumor cells or in the surrounding tissue. Thus, there is a need to identify non-invasive techniques for investigating the pharmacokinetics of boron containing molecules.

MRI (Magnetic Resonance Imaging) instruments utilize nuclear magnetic resonance properties of atoms with nuclear spin to create physiological images. The use of ¹H-MRI, ¹¹B-MRI, ¹⁸F-MRI and ¹³C-MRI as a technique for the evaluation of concentration of p-BPA in tumors and surrounding areas has been previously described. ¹H-MRI of p-BPA was moderately successful in observing the presence of p-BPA in mice by focusing on the aromatic protons; however, even while administering p-BPA at high concentrations, obtaining a sufficient signal-to-noise ratio required an extended scan time, limiting resolution requiring specialized equipment and optimization procedures. Bendel et al., “Optimized ¹H MRS and MRSI methods for the in vivo detection of boronophenylalanine” Magn Reson Med. 53(5):1166-71 (2005). For imaging purposes, the low natural concentrations of ¹³C are also insufficient. Imaging using atomically enriched ¹³C, and ¹⁸F compounds has not been satisfactory because their preparation has provided to be difficult and expensive. Being chemically distinct, ¹⁸F labeled p-BPA may not give a good approximation of the distribution of unfluorinated p-BPA in a particular tissue location. Additionally, subjects may experience undesirable adverse drug reactions caused by exposure to the halogenated derivative. Thus, there continues to be a need to identify efficient non-invasive methods for imaging the distribution of p-BPA in specific tissues of a subject.

### BRIEF SUMMARY OF THE INVENTION

The present invention relates to amino acids, complexes, and compounds comprising deuterium and tritium isotopes preferably alpha deuterated amino acids, polypeptides, antibodies, derivatives and saccharide-amino acid complexes and conjugates. In some embodiments, the invention relates to methods of using compounds comprising deuterium for imaging biochemical concentrations and distributions in mammalian tissues using nuclear magnetic resonance spectroscopy. In some embodiments, the invention relates to the used of said amino acids derivatives and complexes in boron neutron capture therapy. In some embodiments, the present invention relates to the preparation of amino acids, polypeptides, antibodies, derivatives and saccharide complexes/conjugates comprising heavy hydrogen isotopes. In some embodiments, the invention relates to racemizing amino acids starting from compositions of any optical purity. In further embodiments, the invention relates to the preparation of amino acids and their N-acyl counterparts with deuterium incorporated at the alpha carbon.

In some embodiments, the invention relates to a method for of making alpha-deuterated amino acids comprising a) providing i) a composition comprising a substituted or unsubstituted amino acid ii) a carboxylic acid anhydride, iii) a solution comprising deuterium, and iv) a base, b) mixing said composition, said carboxylic acid anhydride, said deuterated solution and said base under conditions such that an alpha-deuterated N-acylated amino acid is formed. In further embodiments, said substituted amino acid is N-acylated. In further embodiments, an alpha-deuterated amino acid is formed after mixing the alpha-deuterated N-acylated amino acid with an acid.

In some embodiments, the invention relates to a method for racemizing amino acids comprising a) providing i) a composition comprising a purified isomer of amino acid or N-acylated amino acid that is enantiomerically pure or racemic or somewhere in between ii) an carboxylic acid anhydride, and iii) a base, b) i) mixing said composition, said carboxylic acid anhydride, and said base under conditions such that a racemized amino acid or N-acylated amino acid is formed. In further embodiments, said amino acid or N-acylated amino acid is selected from the group consisting of alanine, leucine, isoleucine, valine, phenylalanine, tyrosine and p-boronophenylalanine. In further embodiments, said carboxylic acid anhydride is selected from the group consisting of acetic anhydride, propionic anhydride, butyric anhydride, and pentionic anhydride. In further embodiments, said base comprises MX, wherein M is selected from the group consisting of lithium, berylium, sodium, potassium, magnesium, and calcium and X is selected from the group consisting of hydroxide and hydride. In further embodiments, the method entails providing a solution wherein said solution is selected from the group consisting of water, ethanol, propanol, isopropanol, THF, toluene, benzene, and dichloromethane and mixing said composition, said carboxylic acid anhydride, said base, and said solution under conditions such that a solution of racemized N-acylated amino acid is formed. In further embodiments said carboxylic acid anhydride is acetic anhydride. In further embodiments, said base is sodium hydroxide. In further embodiments, said solution is water. In further embodiments, the method further comprises: providing an acid and step of c) mixing said solution of racemized N-acylated amino acid and said acid under conditions such that a racemized amino acid is formed. In further embodiments, said acid is HₙX wherein n is 1 or 2, and X is selected from the group consisting of chloride, bromide, iodide, fluoride, sulfate. In further embodiments, n is 1 and X is chloride. In further embodiments, the method further comprises, after step b)i) and prior to step c), of b)ii) removing said base from said solution of racemized N-acylated amino using an ion exchange resin. In further embodiments, said ion exchange resin is Dowex 50WX4-50.

In some embodiments, the invention relates to a method of making alpha-deuterated amino acids comprising a) providing i) a composition comprising an amino acid or N-acylated amino acid that is enantiomerically pure or racemic or somewhere in between ii) an carboxylic acid anhydride, iii) a solution comprising a heavy isotope of hydrogen, and iv) a base, b) mixing said composition, said carboxylic acid anhydride, said deuterated solution and said base under conditions such that an alpha-deuterated amino acid or an alpha-deuterated N-acylated amino acid is formed. In further embodiments, said amino acid is selected form the group consisting of alanine, leucine, isoleucine, valine, phenylalanine, tyrosine, tryptophan, and p-boronophenylalanine. In further embodiments, said carboxylic acid anhydride is selected from the group but not limited to acetic anhydride, propionic anhydride, butyric anhydride, and pentanoic anhydride. In further embodiments, said base comprises MX, wherein M is selected from the group consisting of tri-n-butyltin, titanium, lithium, berylium, sodium, potassium, magnesium, and calcium and X is selected from the group consisting of deuteroxide and deuteride. In further embodiments, said solution comprising a heavy isotope of hydrogen is a liquid comprising a compound selected from the group consisting of deuterium oxide, methanol-d₃, methanol-d₄, methan(ol-d), 1,1,1,3,3,3-hexafluoro-2-propan(ol-d), butanol-d₁₀, 1,4-dithiothreitol-d₁₀, 2,2,2-trifluoroethan(ol-d), 2,2,2-trifluoroethanol-1,1-d₂, 2,2,2-trifluoroethanol-d₃, 2-propanol-1,1,1,3,3,3-d₆, 2-propanol-d₈, acetic acid-d₄, acetic acid-OD, ethan(ol-d), ethanol-1,1,2,2,2-d₅, ethanol-2,2,2-d₃, ethanol-d₆, ethylene glycol-(OD)₂, ethylene glycol-d₆, imidazole-d₄, formic acid-d₂, tritium oxide, tritium ethanol, tritium propanol, and tritium isopropanol.

In some embodiments, the invention relates to a non-naturally occurring compound or derivative thereof having the following structure: 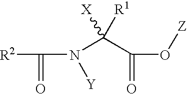
wherein, R¹ is a naturally occurring amino acid side chain, substituted naturally occurring amino acid side chain, hydrogen, deuterium, tritium, borono, halogen, hydroxy, oxo, cyano, nitro, amino, substituted amino, alkylamino, substituted alkylamine, dialkylamino, substituted dialkylamino, alkyl, substituted alkyl, alkoxy, substituted alkoxy, alkylthio, substituted alkylthio, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclalkyl, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)₂ORₐ; Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; R² is alkyl or substituted alkyl; X is deuterium or tritium; Y and Z are the same or different and, at each occurrence, independently hydrogen, deuterium, or tritium.

In some embodiments, the invention relates to a composition comprising a purified isotope form of a compound or derivative thereof having the following structure: 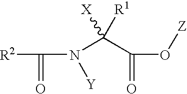
wherein, R¹ is a naturally occurring amino acid side chain, substituted naturally occurring amino acid side chain, hydrogen, deuterium, tritium, borono, halogen, hydroxy, oxo, cyano, nitro, amino, substituted amino, alkylamino, substituted alkylamine, dialkylamino, substituted dialkylamino, alkyl, substituted alkyl, alkoxy, substituted alkoxy, alkylthio, substituted alkylthio, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclalkyl, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ or —S(═O)²ORₐ; Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; R² is alkyl or substituted alkyl; X is deuterium or tritium; Y and Z are the same or different and, at each occurrence, independently hydrogen, deuterium, or tritium.

In some embodiments, the invention relates to a composition comprising a purified isotope form of a compound or derivative thereof having the following structure: 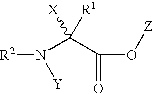
wherein, R¹ is a naturally occurring amino acid side chain, substituted naturally occurring amino acid side chain, hydrogen, deuterium, tritium, borono, halogen, hydroxy, oxo, cyano, nitro, amino, substituted amino, alkylamino, substituted alkylamine, dialkylamino, substituted dialkylamino, alkyl, substituted alkyl, alkoxy, substituted alkoxy, alkylthio, substituted alkylthio, haloalkyl, substituted haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclalkyl, —NRₐR_{b}, —NRₐC(═O)R_{b}, —NRₐC(═O)NRₐNR_{b}, —NRₐ C(═O)OR_{b}—NRₐSO₂R_{b}, —C(═O)Rₐ, C(═O)ORₐ, —C(═O)NRₐR_{b}, —OC(═O)NRₐR_{b}, —ORₐ, —SRₐ, —SORₐ, —S(═O)₂Rₐ, —OS(═O)2Rₐ or —S(═O)₂ORₐ; Rₐ and R_{b} is the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl; R² is hydrogen, deuterium, tritium, alkyl or substituted alkyl; X is deuterium or tritium; and Y and Z are the same or different and, at each occurrence, independently hydrogen, deuterium, or tritium.

In some embodiments, the invention relates to an amino acid with enhanced or complete optical activity (L or D) with deuterium or tritium at the alpha-carbon of the amino acid. In some embodiments, the invention relates to a non-naturally occurring and purified isotopic comprising an amino acid with enhanced or complete optical activity with deuterium or tritium at the alpha-carbon of the amino acid. In some embodiments, the invention relates to a non-naturally occurring and purified isotopic amino acid composition comprising deuterium enriched at the alpha-carbon of the amino acid. In further embodiments, said amino acid composition has enhanced optical activity. In further embodiments, said amino acid is p-BPA.

In some embodiments, the invention relates to a composition comprising a purified isotope form of a compound or derivative thereof having the following structure: 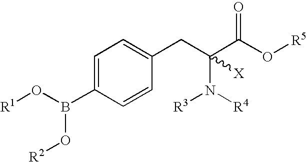
wherein, R¹, R², R³, R⁴, R⁵ are the same or different and, at each occurrence, independently hydrogen, deuterium, tritium and X is deuterium or tritium.

In some embodiment, the invention relates to a non-naturally occurring isotopic form of a compound or derivative thereof having the following structure: 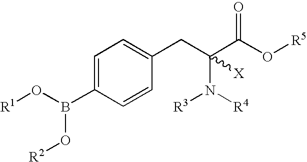
wherein, R¹, R², R³, R⁴, R⁵ are the same or different and, at each occurrence, independently hydrogen, deuterium, tritium and X is deuterium or tritium.

In some embodiments, the invention relates to a non-naturally occurring isotopic composition of a compound or derivative thereof having the following structure:

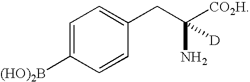

In some embodiments, the invention relates to a non-naturally occurring isotopic composition of a compound or derivative thereof having the following structure:

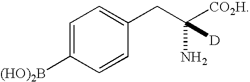

In some embodiments, the invention relates to a method of preparing the of alpha-deutero-p-boronophenylalanine comprising: a) providing: i) (2,2-dicarbethoxy-2-acetamidoethyl)-benzeneboronic acid, ii) a base, ii) deuterium oxide, and iv) an acid; b) mixing said (2,2-dicarbethoxy-2-acetamidoethyl)-benzeneboronic acid, said base, and said deuterium oxide under conditions such that a solution of d₆-(2,2-dicarboxy-2 acetamidoethyl) benzeneboronic acid is produced; c) mixing said solution of d₆-(2,2-dicarboxy-2 acetamidoethyl) benzeneboronic acid with said acid under conditions such that alpha-deutero-p-boronophenylalanine is produced. In further embodiments, said base is MX, wherein M is selected from the group consisting of lithium, berylium, sodium, potassium, magnesium, and calcium and X is selected from the group consisting of hydride, deuteride, hydroxide, and deuteroxide. In further embodiments, said base is sodium deuteroxide. In further embodiments, said acid is selected from the group consisting of HCl, HBr, HI, H₂SO₄, DCl, DBr, DI, and D₂SO₄. In some embodiments, the invention is a method for preparing alpha-deutero-p-boronophenylalanine comprising: comprising a) providing i) N-acylated p-boronophenylalanine ii) an carboxylic acid anhydride, iii) a solution comprising a heavy isotope of hydrogen, iii) a base, and iv) an acid; b) mixing N-acylated p-boronophenylalanine, said carboxylic acid anhydride, said deuterated solution and said base under conditions such that a alpha-deuterated N-acylated p-boronophenylalanine is formed; c) mixing said alpha-deuterated N-acylated p-boronophenylalanine with said acid under conditions such that alpha-deutero-p-boronophenylalanine is formed.

In some embodiments, the invention relates to a use of natural abundance B or ¹⁰B enriched L-alpha-deutero-p-boronophenylalanine as a boron neutron capture therapy agent.

In some embodiments, the invention relates to a use of natural abundance B or ¹⁰B enriched L-alpha-deutero-p-boronophenylalanine as an MRI agent.

In some embodiments, the invention relates to a method of detecting p-boronophenylalanine in mammalian tissue comprising a) providing i) a subject comprising mammalian tissue, ii) a compound comprising a deuterium atom, wherein said compound is alpha-deutero-p-boronophenylalanine, and iii) a magnetic resonance spectrometer configured to detect said deuterium atom; b) administering said compound to said subject; c) detecting said deuterium atom with said magnetic resonance spectrometer in said tissue.

In some embodiments, the invention relates to a method of detecting deuterated compound in mammalian tissue comprising a) providing i) a subject comprising mammalian tissue, ii) a compound comprising a deuterium atom and iii) a magnetic resonance spectrometer configured to detect said deuterium atom; b) administering said compound to said subject; c) detecting said deuterium atom with said magnetic resonance spectrometer in said tissue. In further embodiments, said compound is an amino acid. In further embodiments said amino acid is alpha-deuterated. In further embodiment, invention relates to a method of imaging mammalian tissue comprising the steps above and the further step of transforming deuterium detection data into an image. In further embodiments, said amino acid is selected form the group consisting of alanine, leucine, isoleucine, valine, phenylalanine, tyrosine, tryptophan, and p-boronophenylalanine. In some embodiments, the invention relates to creating polypeptides, proteins, and amino acid sequences having alpha-deuterated amino acids in order to create MRI.

In some embodiments, the invention relates to methods of creating insulin with alpha-deuterated amino acids. In some embodiments, the invention relates to a method of detecting said insulin in mammalian tissue comprising a) providing i) a subject comprising mammalian tissue, ii) a insulin comprising a alpha-deuterated amino acids and iii) a magnetic resonance spectrometer configured to detect said deuterium atoms; b) administering said insulin to said subject; c) detecting said deuterium atom with said magnetic resonance spectrometer in said tissue. In further embodiments, the concentration of insulin in the circulatory system is detected. In some embodiments, the invention relates to creating insulin or insulin conjugated to a saccharide comprising polypeptides sequences having alpha-deuterated amino acids in order to create MRI. In further embodiments, said images are used to diagnose type I or type II diabetes.

In some embodiments, the invention relates to methods of creating antibodies with alpha-deuterated amino acids. In some embodiments, the invention relates to a method of detecting antibodies in mammalian tissue comprising a) providing i) a subject comprising mammalian tissue, ii) a antibody comprising a alpha-deuterated amino acids and iii) a magnetic resonance spectrometer configured to detect said deuterium atoms; b) administering said antibody to said subject; c) detecting said deuterium atom with said magnetic resonance spectrometer in said tissue.

In some embodiments, the invention relates to diagnosis of Alzheimer's disease by using deuterium imaging to identify the presence of Alzheimer's plaques. In further embodiments, the invention relates to antibodies to betaAP comprising alpha-deuterated amino acids that transports across the mammalian blood-brain barrier. In further embodiments, genetically engineered chimeric humanized monoclonal antibodies to betaAP comprising alpha-deuterated amino acids are transported across the blood-brain barrier. In some embodiments, an antibody to betaAP comprising alpha-deuterated amino acids is conjugated to mannose 6-phosphate/insulin-like growth factor 2. In some embodiments, an antibody to betaAP comprising alpha-deuterated amino acids is conjugation to a blood-brain barrier receptor-specific monoclonal antibody transferrin receptor by a streptavidin-biotin linkage.

In some embodiments, the invention relates to a composition comprising a compound selected from the group consisting of: a p-boronophenylalanine-α,α-glucooctitol complex; p-boronophenylalanine-volemitol complex; p-boronophenylalanine-persietol complex; p-boronophenylalanine-α-glucoheptitol complex; p-boronophenylalanine-lactitol complex; p-boronophenylalanine-cellobiitol complex; p-boronophenylalanine-isomaltitol complex; p-boronophenylalanine-maltitol complex; p-boronophenylalanine-aminosorbitol complex; p-boronophenylalanine-aminodulcitol complex; p-boronophenylalanine-dulcitol complex; p-boronophenylalanine-mannitol complex; p-boronophenylalanine-sorbitol complex; p-boronophenylalanine-rhamnitol complex; and p-boronophenylalanine-xylitol complex.

In further embodiments, said p-boronophenylalanine saccharide complex is alpha-deuterated. In further embodiments, the complex is used for treating or preventing cancer. In further embodiments, the complex is used for treating or preventing glioblastoma multiforme. In further embodiments, the deuterated complex is used to image a tissue of a subject with nuclear magnetic resonance spectroscopy. In further embodiments the deuterated complex is used in boron neutron capture therapy.

In some embodiments, the invention relates to a method of detecting and imaging alpha deuterated p-BPA and complexes, determining concentrations of p-BPA in tissues, and administering radiation for boron neutron capture therapy when p-BPA is at its highest concentrations in the tissue such as when p-BPA saccharide complexes penetrate tumors.

In some embodiments, the invention relates to a method of racemizing amino acids comprising a) providing i) an amino acid or N-acylated amino acid ii) a carboxylic acid anhydride, iii) a solution of water, and iv) a base, b) mixing said amino acid or N-acylated amino acid, said solution of water, and said base under conditions providing a basic solution; c) mixing said basic solution with said carboxylic acid anhydride under conditions such that said amino acid or N-acylated amino acid is racemized. In further embodiments, said N-acylated amino acid is p-boronophenylalanine. In further embodiments, said carboxylic acid anhydride is acetic anhydride. In further embodiments, said base is sodium hydroxide.

In some embodiments, the invention relates to a method of making alpha-deuterated amino acids comprising: a) providing i) a substituted or unsubstituted amino acid ii) a carboxylic acid anhydride, iii) a solution of deuterium oxide, and iv) a base, b) mixing said N-acylated amino acid, said solution of deuterium oxide, and said base under conditions providing a basic solution; c) mixing said basic solution with said carboxylic acid anhydride under conditions such that said N-acylated amino acid is racemized. In further embodiments, said N-acylated amino acid is p-boronophenylalanine. In further embodiments, said carboxylic acid anhydride is acetic anhydride. In further embodiments, said base is selected from the group consisting of sodium deuteroxide and sodium deuteride. In further embodiments, said base is presented in greater than one molar equivalent compared to the amino acid. In further embodiments, said base is presented in greater than one and one-half molar equivalents compared to the amino acid. In further embodiments, said base is presented in two or greater than two molar equivalent compared to the amino acid.

In some embodiments, the invention relates to a method for preparing alpha-deutero-p-boronophenylalanine comprising: comprising a) providing i) a substituted or unsubstituted p-boronophenylalanine ii) a carboxylic acid anhydride, iii) a solution comprising deuterium, iv) a base, and v) an acid; b) mixing said p-boronophenylalanine, said carboxylic acid anhydride, said deuterated solution and said base under conditions such that an alpha-deuterated N-acylated p-boronophenylalanine is formed; c) mixing said alpha-deuterated N-acylated p-boronophenylalanine with said acid under conditions such that alpha-deutero-p-boronophenylalanine is formed. In further embodiments, said substituted p-boronophenylalanine is N-acylated.

In some embodiments, the invention relates to a method of detecting p-boronophenylalanine in mammalian tissue comprising a) providing i) a subject comprising mammalian tissue, ii) a compound comprising a deuterium atom, wherein said compound is alpha-deutero-p-boronophenylalanine, and iii) a nuclear magnetic resonance spectrometer configured to detect said deuterium atom; b) administering said compound to said subject; c) detecting said deuterium atom with said nuclear magnetic resonance spectrometer in said tissue. In further embodiments, said alpha-deutero-p-boronophenylalanine is an alpha-deutero-p-boronophenylalanine saccharide complex. In further embodiments, said alpha-deutero-p-boronophenylalanine saccharide complex is selected from the group consisting of: alpha-deutero p-boronophenylalanine-α,α-glucooctitol complex; alpha-deutero p-boronophenylalanine-volemitol complex; alpha-deutero p-boronophenylalanine-persietol complex; alpha-deutero p-boronophenylalanine-α-glucoheptitol complex; alpha-deutero p-boronophenylalanine-lactitol complex; alpha-deutero p-boronophenylalanine-cellobiitol complex; alpha-deutero p-boronophenylalanine-isomaltitol complex; alpha-deutero p-boronophenylalanine-maltitol complex; alpha-deutero p-boronophenylalanine-aminosorbitol complex; alpha-deutero p-boronophenylalanine-aminodulcitol complex; alpha-deutero p-boronophenylalanine-dulcitol complex; alpha-deutero p-boronophenylalanine-mannitol complex; alpha-deutero p-boronophenylalanine-sorbitol complex; alpha-deutero p-boronophenylalanine-rhamnitol complex; and alpha-deutero p-boronophenylalanine-xylitol complex.

In some embodiments, the invention relates to a method comprising a) providing I) a subject diagnosed with a tumor ii) an alpha deuterated p-boronophenylalanine or saccharide complex and b) administering said deuterated 4-boronophenylalaine or saccharide complex to said subject. In further embodiment said tumor is a brain tumor. In further embodiments, said tumor is benign or malignant.

In some embodiments, the invention relates to a method of treating or preventing cancer comprising administering a substituted or unsubstituted alpha deuterated 4-boronphenylalanine or saccharide complex and performing boron neuron capture therapy by exposing said subject to neutron irradiation. In further embodiments, said cancer is glioblastoma multiforme.

### BRIEF DESCRIPTION OF THE DRAWINGS

FIG. 1. Illustration of the racemization of amino acids using acetic anhydride, sodium hydroxide and water.

FIG. 2. Illustration of incorporating deuterium into amino acids using acetic anhydride, sodium deuteroxide and deuterium oxide.

FIG. 3. Illustration of the application of the amino acid racemization to the preparation of racemic p-boronophenylalanine and 2-deutero-p-boronophenylalanine.

FIG. 4. Illustration of alternative method for the preparation of racemic alpha-deutero-para-boronophenylalanine.

FIG. 5. Illustration of deuterated amino acids obtained by disclosed synthetic procedures.

### DETAILED DESCRIPTION OF THE INVENTION

The present invention relates to amino acids, complexes, and compounds comprising deuterium and tritium isotopes preferably alpha deuterated amino acids, polypeptides, antibodies, derivatives and saccharide-amino acid complexes and conjugates. In some embodiments, the invention relates to methods of using compounds comprising deuterium for imaging biochemical concentrations and distributions in mammalian tissues using nuclear magnetic resonance spectroscopy. In some embodiments, the invention relates to the used of said amino acids derivatives and complexes in boron neutron capture therapy. In some embodiments, the present invention relates to the preparation of amino acids, polypeptides, antibodies, derivatives and saccharide complexes/conjugates comprising heavy hydrogen isotopes. In some embodiments, the invention relates to racemizing amino acids starting from compositions of any optical purity. In further embodiments, the invention relates to the preparation of amino acids and their N-acyl counterparts with deuterium incorporated at the alpha carbon.

The term “acid” when used in relation to a substance in a synthetic method means a substance that can act as a proton donor in a solution including, but not limited to, substances that cause a pH and/or pD of less than 6.8 in an aqueous solution.

“Acyl” means an —C(═O)alkyl or —C(═O)aryl group.

“Acyloxy” means —Oacyl.

“Adverse drug reaction” means any response to a drug that is noxious and unintended and occurs in doses for prophylaxis, diagnosis, or therapy including side effects, toxicity, hypersensitivity, drug interactions, complications, or other idiosyncrasy. Side effects are often adverse symptom produced by a therapeutic serum level of drug produced by its pharmacological effect on unintended organ systems (e.g., blurred vision from anticholinergic antihistamine). A toxic side effect is an adverse symptom or other effect produced by an excessive or prolonged chemical exposure to a drug (e.g., digitalis toxicity, liver toxicity). Hypersensitivities are immune-mediated adverse reactions (e.g., anaphylaxis, allergy). Drug interactions are adverse effects arising from interactions with other drugs, foods or disease states (e.g., warfarin and erythromycin, cisapride and grapefruit, loperamide and Clostridium difficile colitis). Complications are diseases caused by a drug (e.g., NSAID-induced gastric ulcer, estrogen-induced thrombosis). The adverse drug reaction may be mediated by known or unknown mechanisms (e.g., Agranulocytosis associated with chloramphenicol or clozapine). Such adverse drug reaction can be determined by subject observation, assay or animal model well-known in the art.

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. The term “higher alkyl” has the same meaning as alkyl but contains from 2 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Cyclic alkyls are also referred to herein as a “homocycles” or “homocyclic rings.” Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkylamino” and “dialkylamino” mean one or two alkyl moiety attached through a nitrogen bridge (i.e., —N-alkyl) such as methylamino, ethylamino, dimethylamino, diethylamino, and the like.

“Alkoxy” means an alkyl moiety attached through an oxygen bridge (i.e.,—O-alkyl) such as methoxy, ethoxy, and the like.

“Alkylthio” means an alkyl moiety attached through a sulfur bridge (i.e., —S— alkyl) such as methylthio, ethylthio, and the like.

“Alkylsulfonyl” means an alkyl moiety attached through a sulfonyl bridge (i.e., —SO₂-alkyl) such as methylsulfonyl, ethylsulfonyl, and the like.

“Alpha-deuterated amino acid” means an amino acid with deuterium bonded to an alpha-carbon, i.e., the carbon between the “amino” nitrogen atom and the “acid” carbonyl carbon atom of the “amino acid”.

“Amino acid” means an substituted or unsubstituted organic compound containing an amino group (NH₂), a carboxylic acid group (COOH), where the carboxyl group (COOH) and the amino group (NH₂) are attached to the same carbon at the end of the compound. The 20 amino acids commonly found in animals are alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. More than 100 less common amino acids also occur in biological systems, particularly in plants.

“Aryl” means an aromatic carbocyclic moiety such as phenyl or naphthyl.

“Arylalkyl” means an alkyl having at least one alkyl hydrogen atoms replaced with an aryl moiety, such as benzyl, —(CH₂)₂phenyl, —(CH₂)₃phenyl, —CH(phenyl)₂, and the like.

The term “base” when used in relation to a substance for a synthetic method means a substance that can act as a proton acceptor in a solution including, but not limited to, substances that cause a pH and/or pD of more than 7.2 in an aqueous solution.

“Boron neutron capture therapy” means administration of a boron containing composition to a subject followed by neutron irradiation of specific tissues of the subject. “Boron neutron capture therapy agent” means the boron composition used in boron neutron capture therapy.

“Carboxylic acid anhydride” means a compound containing two substituted or unsubstituted acyl groups bridged by an oxygen atom. Carboxylic acid anhydrides can be symmetrical or asymmetrical.

In the context of certain embodiments, a “complex” means a conjugate formed by an association of atoms in a solution through non-covalent bonds and/or coordinate covalent bonds. A saccharide complex contains a saccharide compound. For example, L-p-boronophenylalanine-fructose complex contains the saccharide, fructose, which in solution is conjugated to L-p-boronophenylalanine in an association of atoms.

“Conjugate” means a compound that has been formed by the joining of two or more compounds by covalent and/or non-covalent bonds. In some embodiment, the conjugate is an antibody joined to a polypeptide sequence comprising alpha-deuterated amino acids. In further embodiments, the antibody contains alpha-deuterated amino acids.

“Complete optical activity” means the ability of a composition composed entirely of an asymmetric compound to rotate the orientation of planar polarized light a maximum amount for that particular asymmetric compound.

“Deuterium MRI agent” means a compound used for administration of the compound to a subject followed by imaging by detecting nuclear magnetic resonances of deuterium nuclei in tissues of the subject.

“Diastereomers” are stereoisomers that are not enantiomers (i.e., mirror images of each other). The term is intended to include salts formations (e.g., tartaric acid salts). Diastereomers can have different physical properties and different reactivity.

“Enantiomeric excess” (ee) refers to the products that are obtained by a synthesis comprising an enantioselective step, whereby a surplus of one enantiomer in the order of at least about 52% ee is yielded.

“Enhanced optical activity” means the ability of a composition to rotate the orientation of planar polarized light to some degree between no ability and complete optical activity. Although differing in geometric arrangement, enantiomers possess identical chemical and physical properties. Since each type of enantiomer affects polarized light differently, optical activity can be used to identify which enantiomer is present in a sample and its purity. Optical activity may be measured by, but not limited to, two methods: optical rotation, which observes a sample's effect on the velocities of right and left circularly polarized light beams; and circular dichroism, which observes a sample's absorption of right and left polarized light.

“Halogen” means fluoro, chloro, bromo and iodo.

“Haloalkyl” means an alkyl having at least one hydrogen atom replaced with halogen, such as trifluoromethyl and the like.

“Heavy isotope of hydrogen” means atoms of hydrogen with the same atomic number but with neutrons. Heavy isotopes of hydrogen include deuterium and tritium. Different isotopes of a given element also have the same number of electrons and the same electronic structure. Because the chemical behavior of an atom is largely determined by its electronic structure, isotopes exhibit nearly identical chemical behavior. The primary exception is that, due to their larger masses, heavier isotopes tend to react somewhat more slowly than lighter isotopes of the same element. (This phenomenon is termed the kinetic isotope effect). This “mass effect” is pronounced for protium (¹H) vis-à-vis deuterium (²H), because deuterium has twice the mass of protium. For heavier elements the relative mass difference between isotopes is less, and the mass effect is usually negligible.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocyclic ring”) means a 4- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂morpholinyl, and the like.

“Homocycle” (also referred to herein as “homocyclic ring”) means a saturated or unsaturated (but not aromatic) carbocyclic ring containing from 3-7 carbon atoms, Such as cyclopropane, cyclobutane, cyclopentane, cyclohexane, cycloheptane, cyclohexene, and the like.

“Isomers” means any of two or more substances that are composed of the same elements in the same proportions but differ in the three dimensional arrangement of atoms including enantiomeric (i.e., mirror images) and diastereomeric isomers.

The term “manage” when used in connection with a disease or condition means to provide beneficial effects to a patient being administered with a prophylactic or therapeutic agent, which does not result in a cure of the disease. In certain embodiments, a patient is administered with one or more prophylactic or therapeutic agents to manage a disease so as to prevent the progression or worsening of the disease.

“Methylene” means —CH₂—.

“Natural abundance” of an isotope means the average amount of atom isotopic distribution of a particular elemental composition found normally in nature. Isotopes are different forms of the same element, with nuclei that have the same number of protons but different numbers of neutrons. Isotopes are distinguished from each other by giving the combined number of protons and neutrons in the nucleus. Thus isotopes of a given element may have slightly different physical properties. All elements have isotopes. Collectively, the isotopes of the elements form the set of nuclides. A nuclide is a particular type of atomic nucleus, or more generally an agglomeration of protons and neutrons. In symbolic form, the number of nucleons (protons and neutrons) is denoted as a superscripted prefix to the chemical symbol (e.g., ²H or D for deuterium).

The term “derivative” when used in relation to a chemical compound refers to a similar structure that upon administration to the recipient is capable of providing, directly or indirectly, the function said chemical compound is disclosed to have.

As used herein, the terms “prevent” and “preventing” include the prevention of the recurrence, spread or onset. It is not intended that the present invention be limited to complete prevention. In some embodiments, the onset is delayed, or the severity of the disease is reduced.

As used herein, the terms “purified isomer” and “purified isomer composition” are meant to indicate a composition (e.g. derived from a racemic mixture or synthesized de novo) wherein one isomer has been enriched over the other, and more preferably, wherein the other isomer or isomers represents less than 10%, and more preferably less than 7%, and still more preferably, less than 2% of the preparation.

A “tumor” means an abnormal mass of tissue growth that may be classified as benign or malignant.

Compositions comprising “purified isotopes” of compounds in accordance with the invention are enriched by 1% more than the natural abundance of a particular atomic isotope at a particular location of the molecule and preferably are enriched to more than 90% at the location of the molecule. The location of enrichment is expressly indicated by designation of the isotope (e.g., ²H or D for deuterium). An alpha-deuterated amino acid is enriched with deuterium attached to the carbon between the amino and carboxyl group, i.e., alpha-carbon. Deuterium has a natural abundance of 0.0155% compared to hydrogen. Thus, a composition comprising an alpha-deuterated amino acid in which the deuterium attached to the alpha-carbon has abundance greater than 1.0155% would be a purified isotope composition.

It is to be understood that references herein to “impurities” are to be understood as to include unwanted reaction products that are not atomic isotopes or isomers formed during synthesis and also does not include residual solvents remaining from the process used in the preparation of the composition or excipients used in pharmaceutical preparations.

The expression “essentially free” of a molecule means that the molecule is present in a composition only as an unavoidable impurity. “Saccharide” means a sugar or substituted sugar exemplified by but is not limited to 2,3-dideoxyhex-2-enopyranoside, 2,3-desoxy-2,3-dehydroglucose, 2,3-desoxy-2,3-dehydroglucose diacetate, glucoside, glucoside tetraacetate, mannoside, mannoside tetraacetate, galactoside, galactoside tetraacetate, alloside, alloside tetraacetate, guloside, guloside tetraacetate, idoside, idoside tetraacetate, taloside, taloside tetraacetate, rhamnoside, rhamnoside triacetate, maltoside, maltoside heptaacetate, 2,3-desoxy-2,3-dehydromaltoside, 2,3-desoxy-2,3-dehydromaltoside pentaacetate, 2,3-desoxymaltoside, lactoside, lactoside tetraacetate, 2,3-desoxy-2,3-dehydrolactoside, 2,3-desoxy-2,3-dehydrolactoside pentaacetate, 2,3-desoxylactoside, glucouronate, N-acetylglucosamine, fructose, sorbose, ribose, galactose, glucose, mannose, 2-deoxygalactose, 2-deoxyglucose, maltulose, lactulose, palatinose, leucrose, turanose, lactose, maltose, mannitol, sorbitol, dulcitol, xylitol, erythitol, threitol, adonitol, arabitol, rhamnitol, talitol, 1-aminodulcitol, 1-aminosorbitol, isomaltitol, cellobiitol, lactitol, maltitol, volemitol perseitol, glucoheptitiol, alpha,alpha-glucooctitiol including polysaccharides, carbohydrates, and polyols (i.e., compounds having a large ratio of primary and secondary protected or unprotected hydroxyl groups where if unprotected have a ratio of hydrogen to carbon atoms near 2:1). Saccharides can be derivatized with molecular arrangements that facilitate synthesis (i.e., contain a protecting group, e.g., acetyl group). Saccharides can be derivatized to form prodrugs.

“Solution” means a heterogeneous or homogeneous mixture of two or more substances, which may be solids, liquids, gases, or a combination of these.

“Subject” means any animal, preferably a human patient, livestock, or domestic pet.

The term “substituted” as used herein means any of the above groups (i.e., alkyl, aryl, arylalkyl, heteroaryl, heteroarylalkyl, homocycle, heterocycle and/or heterocyclealkyl) wherein at least one hydrogen atom is replaced with a substituent. In the case of an oxo substituent (“═O”), two hydrogen atoms are replaced. When substituted, one or more of the above groups are “substituents.” Substituents within the context of this invention include halogen, deuterium, tritium, borono, hydroxy, oxo, cyano, nitro, amino, alkylamino, dialkylamino, alkyl, alkoxy, alkylthio, haloalkyl, aryl, arylalkyl, heteroaryl, heteroarylalkyl, heterocycle and heterocyclealkyl, as well as a saccharide, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. In addition, the above substituents may be further substituted with one or more of the above substituents, such that the substituent substituted alky, substituted aryl, substituted arylalkyl, substituted heterocycle or substituted heterocyclealkyl. Rₐ and R_{b} in this context may be the same or different and independently hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl. In the context of certain embodiments, a compound may be described as “unsubstituted” meaning that the compound does not contain extra substituents attached to the compound. An unsubstituted amino acid refers to the chemical makeup of the amino acid without extra substituents, e.g., the amino acid does not contain a carboxy terminal or amino terminal protecting group(s). For example, unsubstituted proline is the proline amino acid even though the amino group of proline may be considered disubstituted with alkyl groups.

As used herein, the terms “treat” and “treating” are not limited to the case where the subject (e.g. patient) is cured and the disease is eradicated. Rather, the present invention also contemplates treatment that merely reduces symptoms, and/or delays disease progression.

To “modify” a compound means to either add a new chemically bonded atom to said compound, eliminate an atom or group or atoms from the compound, and/or reducing or oxidizing the atomic hybridization state (i.e., sp² to an sp³, reduction, or sp³ to an sp, oxidation) of an atom or group of atoms in the compound.

“Nuclear magnetic resonance spectrometer” means any instrument designed to allow the observation of nuclei as they relax from resonance (i.e., excites the nuclei and then observes the signal as the energy of the nuclei decays back to the ground state) including, but not limited to, those instruments that are used to create images (e.g., but not limited to, MRI instruments).

Neutron capture therapy (NCT) is a binary system for treating tumors in which a chemical agent and thermal or isothermal neutrons are directed to a tumor where they combine to release a lethal dose of radiation to the cell. Boron neutron capture therapy (BNCT) is a form of radiochemotherapy that is becoming increasingly important for the treatment of gliomas, malignant melanomas, and other forms of cancer. BNCT involves administration of a boron compound (¹⁰B) followed by neutron irradiation of the tumor cells or organ. The boron captures a neutron, which results in the release of ionizing helium and lithium ions that are highly damaging and usually lethal to the host cell. For BNCT to be successful, a large number of ¹⁰ B atoms must be localized on or preferably within the target cells, and a sufficient number of thermal neutrons must reach and be absorbed by the ¹⁰B atoms to sustain a lethal reaction. Targeted delivery of boron to tumors is a prerequisite for successful BNCT.

The advantage of BNCT, namely the potential to selectively deliver radiation to a tumor, presents perhaps the greatest challenge. Since the range of the alpha particle is limited to approximately one cell diameter, cells with insufficient amounts of boron will only receive radiation doses similar to those received unavoidably by normal tissues. Cells are most resistant to conventional radiation if they are non-cycling, metabolically inactive, or hypoxic; for the same reason, these same cells may prove most difficult to load with sufficient amounts of boron. A variety of strategies may have to be employed to overcome this problem, e.g., prolonged continuous compound infusions, fractionated infusion and treatment, blood-brain barrier (BBB) disruptions, etc.

In order to achieve adequate tumor control, it is desirable to use doses of a magnitude that will bring the dose to normal tissues close to tolerance levels. The prediction of tolerance dose (TD) in BNCT is extraordinarily difficult due to the complex mix of high and low linear-energy-transfer (LET) radiations whose constituents change rapidly and at different rates with depth in tissue (the adventitious radiation in normal tissues include fast neutrons, protons from nitrogen capture reactions, gammas, and boron dose from normal tissue boron uptake). The possibility that a boron compound may be taken up more avidly by critical regions of the brain favoring a certain metabolic pathway must be considered for every compound in combination with its delivery technique in evaluating its suitability for BNCT use.

Glioblastoma Multiforme (GBM) is one of the most malignant and therapy resistant tumors known. It is usually fatal because of regrowth or resistance of the primary tumor. It is rare to see metastases from GBM; however, its growth pattern can lead to involvement of regions of the brain at some distance from the primary tumor. This fact initially led to the concept of whole brain irradiation. However, tumor control could rarely be achieved within the limits of tolerance of normal brain tissue, and clinically significant recurrence typically first appeared within 2 cm of the primary tumor. Dose escalation studies to more restricted brain volumes with conformal, proton, or brachytherapy techniques led to only minor improvements in treatment outcomes in selected patient groups. Fast neutron therapy (a form of high LET radiation) was interpreted as capable of tumor control, but not within limits of normal brain tolerance. The rationale for targeting BNCT to GBM is based on the fact that GBM is a highly aggressive and resistant tumor that recurs almost exclusively in or near the primary site, and is highly resistant to all treatment approaches. Delivery of thermal neutrons to the tumor in adequate fluencies, but without exceeding normal tissue tolerance, is possible in brain sites. For compounds that freely cross the barrier like BPA, uptake in dividing brain tumor cells leads to increased selectivity in an environment of non-cycling cells. Finally, the depth of penetration required of an epithermal beam to deliver a successful BNCT treatment is now achievable for brain tumors.

Neutron beam design and development in BNCT has reached a level of sophistication with multiple reactor beams, and potentially several non-reactor neutron beams are available for clinical use. Reactor and non-reactor neutron sources, like accelerator-based neutron sources, inherently differ in the energy spectrum with which the neutrons are born. In-phantom beam optimization within a specific institution generally entails the use of a standard phantom and irradiation geometry. In many instances the beam design (filter assembly) optimization and evaluation parameters define, in some form, the usefulness of the beam in treating a tumor with a certain boron compound. The normal tissue biologically weighted dose versus depth for a given beam will be significantly different in magnitude and its mix when evaluated for one compound versus the other. When it comes to assumptions for the tumor it is unknown if these assumptions reflect the reality in a biological system and a probably heterogeneous tumor. The cellular microdistribution of boron within a tumor will be different from patient to patient, and change with boron compound used, resulting in different tumor radiobiological effectiveness for the boron reaction.

The ideal boron carrying compound would not accumulate in normal tissues and would very selectively target at least the surface of all tumor cells or, even better, the tumor DNA, for an additional order of magnitude increase in effect. This highly selective compound would also penetrate the blood-brain barrier to reach even microscopic extensions of tumor and would clear from normal brain by the time radiation is delivered.

One of the compounds currently being used in BNCT is boronophenylalanine (BPA). How this compound is metabolized and what constitutes the biochemical basis of tumor accretion is still not completely understood. The uptake of any compound is dependant on the delivery method and therefore requires clinical trials to optimize the best delivery method. The process of evaluation of a new boron compound for use in BNCT involves multiple steps. First, in vitro studies are carried out to screen compounds. Compounds that lead to significant cell-killing at concentrations that can potentially be achieved in tumors are identified as promising. Promising compounds are evaluated for toxicity and efficacy in in vivo models with small animals with implanted tumors. Next, promising compounds must be evaluated in large animals for toxicity, pharmacokinetics, uptake, best delivery method, safety, and efficacy. Compounds that still show promise must be tested for uptake in human subjects.

One of the challenges is patient recruitment itself. A compound uptake clinical trial to study safety and toxicity of a compound in patients first requires the administration of escalating doses of the compound to brain tumor patients. These studies do not involve any neutron irradiation. Only the pharmacokinetics and uptake of the compound are studied. Recruitment is difficult because the patient selection criteria for these studies reduce the eligible patient population. In addition, the strongest reason why patients participate in clinical trials is the perceived hope of some clinical benefit from the clinical trial. In the case of the compound uptake study for a binary therapy like BNCT, the compound uptake study provides no possibility whatsoever of benefit to the patient. Moreover, investigations optimizing compound delivery by different methods, e.g., intra-arterial delivery and blood-brain barrier (BBB) disruptions, result in added clinical risk to the patient, without clinical benefit. Hence, given a choice between participating in a BNCT compound uptake study and other competing clinical trials with perceived clinical benefits; patients often choose to not participate in the BNCT compound uptake trials.

The data collected in compound uptake studies typically consist of blood samples (drawn at certain intervals after the commencement of compound infusion to well after the compound infusion is completed), urine, and tissue samples from tumor and the surgical resection margins. The goals of a compound uptake study are partly to monitor toxicity of the compound, but more importantly, to perform pharmacokinetic studies to predict the uptake and clearance of the compound from the blood and tumor. The availability of blood samples is usually not a problem. However, the tumor samples are available only at the time of surgery for a given patient. A large cohort of patients is needed to get a number of tumor data points for different time intervals from end of infusion for any given compound dosing scheme (i.e., prolonged infusion, etc.). To add to the complications, there is presently no reasonable way of measuring cell-to-cell variations in the distribution of ¹⁰B within the tumor, and assumptions of uniform ¹⁰B distribution within the heterogeneous tumor are simply speculative.

Tumor metabolism is coupled to tumor blood flow (TBF) and both metabolism and blood flow may be determinants of tumor response to treatment. Since NMR has been used to monitor tumor metabolism non-invasively, development of NMR-based methods for TBF measurement was motivated by the desire to examine the roles tumor metabolism and blood flow may play as determinants of therapeutic response. The concept of using deuterated water as an NMR-detectable, flow-limited tracer for the measurement of tissue blood flow (or capillary perfusion) was introduced in 1987. Ackerman et al., Proc. Natl., Acad. Sci., USA 84, 4099-4102 (1987). Since that time, methods have been devised using both spectroscopic and imaging detection for TBF measurement based on either clearance or uptake of deuterated water.

Boronophenylalanine was initially proposed as a boron delivery drug for boron neutron capture therapy of malignant melanoma because it was postulated that this amino acid would selectively accumulate in melanoma cells by mimicking phenylalanine. An amino acid precursor of melanin, boronophenylalanine has been shown to be selectively taken up by melanoma cells. However, intravenous infusion of BPA is not widely used because BPA exhibits poor water solubility at physiological pH. One method for increasing BPA solubility is the use complexes with saccharides as described in U.S. Pat. No. 6,169,076 and references therein, hereby incorporated by reference. However, there continues to be a need to discover boronophenylalanine derivatives and complexes with desirable solubility characteristics capable of specific physiological interactions for targeting particular cells for neutron capture therapy.

Naturally occurring boron is approximately 3 to 1 mixture of ¹¹B and ¹⁰B, respectively. The use of ¹¹B MRI is undesirable for neutron capture therapy because it is preferred to use p-BPA enriched with ¹⁰B in order to maximize the effects of the therapy. Detecting ¹⁰B is problematic because the signal to noise (S/N) spectroscopic detection sensitivity of ¹⁰B nuclear magnetic resonance has been reported to be less than 5 times that of ¹H. Bendel & Sauerwein, “Optimal detection of the neutron capture therapy agent borocaptate sodium (BSH): a comparison between ¹H and ¹⁰B NMR” Med Phys. 28(2):178-83 (2001). To this end, an embodiment of the current invention attempts to incorporate deuterium or tritium into boronophenylalanine at the alpha-carbon of the amino acid, and use deuterium or tritium magnetic resonance imaging of the alpha-deuterium/tritium boronophenylalanine to determine concentrations of the compound in mammalian tissues preferably brain tumors.

It is contemplated that the carbohydrates used for complexing p-BPA including saccharides can take on either a pyranose or furanose ring form and include, but are not limited to, fructose, sorbose, ribose, galactose, glucose, mannose, 2-deoxygalactose, 2-deoxyglucose, maltulose, lactulose, palatinose, leucrose, turanose, lactose, maltose, mannitol, sorbitol, dulcitol, xylitol, erythitol, threitol, adonitol, arabitol, rhamnitol, talitol, 1-aminodulcitol, 1-aminosorbitol, isomaltitol, cellobiitol, lactitol, maltitol, volemitol perseitol, glucoheptitiol, alpha,alpha-glucooctitiol.

For fructose and sorbose, or when a large excess of another carbohydrate or polyol is used, the p-BPA remains in solution at a pH of 7.4, and approximately 90% of the p-BPA is present as its sodium carboxylate, with the remainder as its free acid. A large excess of carbohydrate or polyol is preferred when pK is small; however, for complexes with a pK>about 3 a large excess in not need. The ratio of free p-BPA to p-BPA:carbohydrate complex can be determined by integration of the aromatic protons resonances (p-BPA, 7.73 and 7.33 ppm; p-BPA-carbohydrate complex, 7.5 and 7.2 ppm) in the 1H-NMR spectrum using D₂O buffered to physiological pH as the solvent. For example, L-p-boronophenylalanine fructose complex has a pK of 3.2 while the meso-erythitol complex has a pK of 1.0, the threitol complex has a pK of 1.3, the adonitol complex has a pK of 1.7, the arabitol complex has a pK of 2.3, the rhamnitol complex has a pK of 2.3, the xylitol complex has a pK of 2.5, the talitol complex has pK of 2.4, the dulcitol complex has a pK of 2.7, the mannitol complex has a pK of 2.7, the 1-aminodulcitol complex has a pK of 2.9, the sorbitol complex has a pK of 3.2, the 1-aminosorbitol complex has a pK of 3.4, the isomaltitol complex has a pK of 3.4, the cellobiitol complex has a pK of 3.5, the lactitol complex has a pK of 3.7, the maltitol complex has a pK of 3.9, the volemitol complex has a pK of 2.9, the perseitol complex has a pK of 3.1, the glucoheptitol complex has a pK of 3.9, and the alpha,alpha-glucooctitol complex has a pK of 4.1.

Deuterium and tritium may be introduced at the alpha-carbon during the synthesis of D,L-p-BPA by substituting D₂0 or T₂0 for water as the solvent during decarboxylation of (2,2-dicarboxy-2-acetamidoethyl) benzeneboronic acid as disclosed in Snyder et al., “Synthesis of Aromatic Boronic Acids. Aldehydo Boronic Acids and a Boronic Acid Analog of Tyrosine,” J. Am. Chem. Soc. 80: 835-838 (1958). Preferably, it is desirable to carry out the hydrolysis of (2,2-dicarbethoxy-2-acetamidoethyl)-benzeneboronic acid in basic D₂O and first isolate the bis carboxylic acid (2,2-dicarboxy-2-acetamidoethyl) benzeneboronic acid. Decarboxylation of the bis carboxylic acid can be carried out by dissolving it in fresh D₂O and HCl with heating.

N-Acetyl-D-p-BPA is a byproduct of the commercial synthesis of the L-p-BPA as the acyl group is preferentially enzymatically removed during isolation of the L isomer. It is desirable to racemized the unwanted D isomer obtained to make it available as raw material for the resolution processes. U.S. Pat. Nos. 6,080,887 and 4,602,096 disclose methods of racemizing N-acetyl-amino carboxylic acids by heating them in the presence of acetic anhydride. Japanese Patent S51-138603 disclosed that N-acetyl-D(L)-alpha-amino carboxylic acid salts in aqueous solution at slightly acid or neutral pH (6-7), can be racemized at a temperature of between 40-90° C. Alpha-hydrogen atoms of amino acids with alkyl side chains such as alanine and phenylalanine are know to be stable under acidic conditions, thus these amino acids do not easily incorporate deuterium and tritium under at the alpha-carbon under acid conditions. It was unclear at the time of performing the experiment whether addition of deuterated sodium hydroxide, and deuterium oxide and acetic anhydride would result in a racemized product because it was thought that with the large amount of water, hydroxide ion would decompose the excess anhydride. This was further complicated by the potential deutero mass effect of using deuterated agents.

Racemization of particular amino acids does not occur at elevated temperatures under strongly acid conditions (e.g., in a 6N aqueous HCl solution). Alpha-hydrogen atoms of amino acids with alkyl side chains such as alanine and phenylalanine are know to be stable under acidic conditions, thus these amino acids do not easily incorporate deuterium and tritium under at the alpha-carbon under acid conditions. See Manning “Determination of D- and L-Amino Acid Residues in Peptides. Use of Tritiated Hydrochloric Acid to Correct for Racemization during Acid Hydrolysis” J. Am. Chem. Soc. 92(25): 7449-7454 (1970). Rittenberg et al., “Deuterium as an indicator in the study of intermediary metabolism” J. Chem. Biol. 125: 1-12 (1938). U.S. Pat. No. 3,213,106 (1965) discloses that a number of amino acids undergo racemization at high temperatures under weakly acidic or basic aqueous solutions. However, phenylalanine is known to proceed slowly in acid conditions, and decompose under basic conditions.

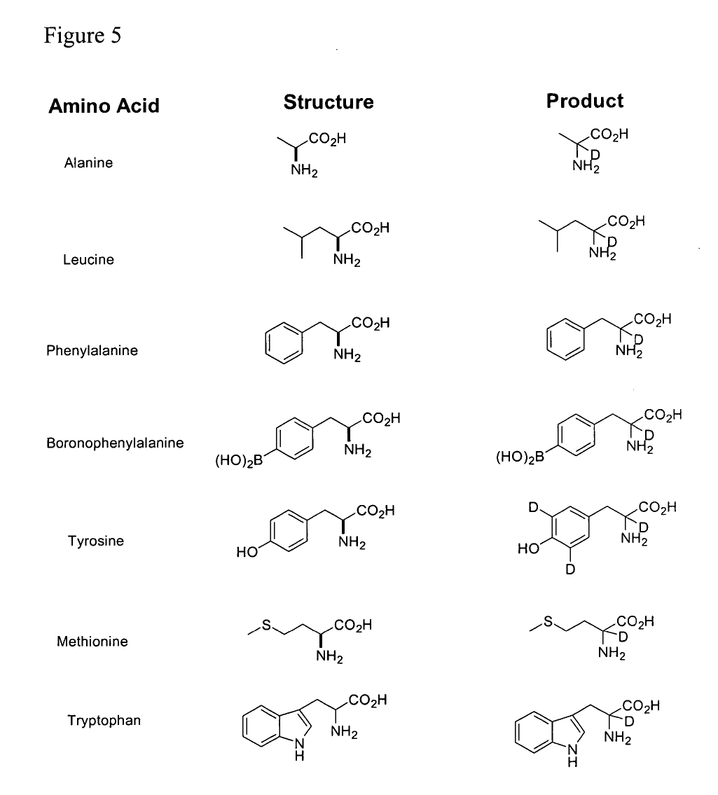

One embodiment of the current invention utilizes deuterium oxide, sodium deuteride, and acetic anhydride for the incorporation of deuterium at the alpha-carbon of amino acids. One embodiment of the current invention is the incorporation of deuterium at the alpha carbon of N-acetyl-p-BPA by exposing the compositions of pure or mixed D and L isomers of N-acetyl-p-BPA to acetic anhydride, sodium deuteride, and deuterium oxide under conditions that provided N-acetyl-alpha-deuterated-p-BPA. See FIG. 5. One embodiment of the current invention is the incorporation of deuterium at the alpha carbon of p-BPA by exposing the compositions of pure or mixed D and L isomers of p-BPA to sodium deuteride, and deuterium oxide under conditions that alpha-deuterated-p-BPA is formed. N-acetyl-alpha-deuterated-p-BPA is transformed to the free amino acid by enzymatic reaction or acidic or basic hydrolysis.

Deuterium oxide and the dilution effect that its presence has in the reaction helps to eliminate (we have never detected any peptide formation) side products allowing for milder conditions (reaction temps 70 C or less). Also, most amino acids are water soluble, and are less likely soluble in neat acetic anhydride. If you add acetic anhydride to a sea of water and some base, one would expect the hydrolysis of the acetic anhydride. It is surprising that the amino acid racemizes under these conditions. No heavy metals are used. Heavy metals are an anathema in drug preparation.

The both the incorporating deuterium either during the initial manufacturing process or during by racemization (including racemization of compositions comprising enantiomerically pure D isomer) using D₂O provides an efficient process for the production of alpha deuterated L-p-BPA.

In one embodiment of the invention is a method of introducing deuterium (²H) preferably in greater than 98% isotopic purity at the alpha-carbon of amino acids, peptides, and derivatives and using the alpha deuterated amino acids, peptides, and derivatives for detection of their pharmacological distribution inside a subject by ²H-MRI. Penetration of alpha-deuterated para-boronophenylalanine is monitored using deuterium magnetic resonance imaging (²H MRI). For example, by administering to a subject alpha-deuterated L-p-BPA, the problems regarding sensitivity and the ability to make accurate measurements of BPA concentrations in particular tissues of the brain by ²H-MRI is overcome. Additionally, this technique allows the inventors to identify particular saccharide complexes of BPA that specifically direct boronated agents to particular tissue types.

U.S. Pat. No. 5,042,488 “Methods Employing Deuterium for Obtaining Direct, Observable Deuterium Magnetic Resonance Images In vivo and in situ” (1991), hereby incorporated by reference, and Bogin et al., “Parametric Imaging of Tumor Perfusion with Deuterium Magnetic Resonance Imaging” Microvascular Research 64, 104-115 (2002) and references cited therein describe methods for creating deuterium magnetic resonance images in animals. Sharf et al., Proc Natl Acad Sci U S A. 1998 April 14;95(8):4108-12 describe the use of ²H Double-quantum filtering (DQF) MRI for the histological imaging of blood vessels.

D₂O was purchased from Cambridge Isotopes Laboratories and the deuterium content assessed as 99%. The extent of deuterium incorporation and the purity of the product are determined by ¹H-NMR. The optical activity is assessed either by HPLC using a chiral mobile phase (such as 3 mM L-proline and 1 mM copper acetate in water in conjunction with a octadecylsilane column) or by measuring its optical rotation with a polarimeter.

Insulin is a polypeptide hormone that is produced in the beta cells of the islets of Langerhans situated in the pancreas of all vertebrates. Insulin is secreted directly into the bloodstream where it regulates carbohydrate metabolism, influences the synthesis of protein and of RNA, and the formation and storage of neutral lipids. Insulin promotes anabolic processes and inhibits catabolic ones in muscle, liver and adipose tissue. Human insulin was among the first commercial health care products produced by recombinant technology.

A number of different processes for the biosynthetic production of polypeptides including human insulin are known. Typically, the DNA strand coding insulin or pro-insulin, a modified form hereof, or the A and B chains of insulin separately, is inserted into a replicable plasmid containing a suitable promoter. By transforming this system into a given host organism a product can be produced which can be converted into authentic human insulin. In some embodiments, the invention relates to methods of producing insulin with alpha-deuterated amino acids in E. coli cells which express insulin or insulin fusion proteins. The cells may be grown in media comprising enriched alpha-deuterated amino acids. Fusion protein may contain pro-sequence which may be cleaved to give insulin by a proteolytic enzyme or the fusion protein may contain a non-naturally occurring cleavage site. Generation of human insulin and pro-insulin are describe in U.S. Pat. No. 5,202,415, U.S. Pat. No. 5,460,954 U.S. Pat. No. 5,925,461, U.S. Pat No. 6,001,004, and U.S. Pat. No. 6,348,327, all hereby incorporated by reference.

The brains of people with Alzheimer's disease contain abnormal tangles and deposits of plaques. It is difficult to observe Alzheimer's plaques. A principal component of these plaques is a protein fragment called beta-amyloid peptide (betaAP). The presence of betaAP in the brain induces the formation of the Alzheimer's plaques. BetaAP is formed when a protein produced in the brain, called the amyloid precursor protein (APP), is cleaved by enzymes in the brain. An enzyme called betasecretase cuts the APP molecule between amino acids, releasing a larger protein fragment called the amino terminal fragment (ATF-betaAPP); and an enzyme called gamma-secretase releases the smaller betaAP fragment. Humans who do not develop Alzheimer's disease break down APP in a manner that does not produce significant amounts of betaAP.

In some embodiments, the invention relates to diagnosis of Alzheimer's disease by using deuterium imaging to identify the presence of Alzheimer's plaques. In further embodiments, the invention relates to antibodies to betaAP comprising alpha-deuterated amino acids capable of transporting across mammalian/primate blood-brain barrier. In further embodiments, the invention relates to genetically engineered chimeric humanized monoclonal antibodies to betaAP comprising alpha-deuterated amino acids capable of crossing the blood-brain barrier of a primate. Deuterium imaging is conducted to diagnosis the presence of Alzheimer's plaques. Monoclonal antibodies are produced from cells grown on media/culture containing alpha-deuterated amino acids. Methods are appropriately modified as described in Coloma et al., “Transport across the primate blood-brain barrier of a genetically engineered chimeric monoclonal antibody to the human insulin receptor” Pharm Res. 17(3):266-74 (2000) and references cited therein.

In some embodiments, the invention relates to direct injection of betaAP antibodies having alpha-deuterated amino acids into the cerebrospinal fluid or osmotic blood-brain barrier disruption causing blood circulating betaAP antibodies having alpha-deuterated amino acids to pass the blood brain barrier. Deuterium imaging is conducted to diagnosis the presence of Alzheimer's plaques.

In some embodiments, an antibody to betaAP comprising alpha-deuterated amino acids is conjugated to a blood-brain barrier receptor-specific monoclonal antibody to transferrin receptor by a streptavidin-biotin linkage. This is accomplished using methods appropriately modified as provided in Zhang and Pardridge “Delivery of beta-Galactosidase to Mouse Brain via the Blood-Brain Barrier Transferrin Receptor” Journal of Pharmacology And Experimental Therapeutics (2005). The conjugate is injected intravenously in subjects, and deuterium imaging is conducted to diagnosis the presence of Alzheimer's plaques.

In some embodiments, an antibody to betaAP comprising alpha-deuterated amino acids is conjugated to mannose 6-phosphate/insulin-like growth factor 2 using procedures appropriately modified as described in Vogler et al., “Overcoming the blood-brain barrier with high-dose enzyme replacement therapy in murine mucopolysaccharidosis VII” Proc. Natl. Acad. Sci. USA 102, 14777-14782 (2005). The conjugate is injected intravenously in subjects, and deuterium imaging is conducted to diagnosis the presence of Alzheimer's plaques.

### EXAMPLE 1

### Racemization of Amino Acids with the Use of Acetic Anhydride, Sodium Hydroxide and Water

Racemization occurs if an amino acid or N-acyl analogs of any optical purity is dissolved in 1.5 L water per mole amino acid and 2.5 molar equivalents of NaOH. Once the amino acid is dissolved, 7.5 molar equivalents of acetic anhydride added and the solution is warmed to 80-90° C. for 3 hour. The warmed solution is then cooled to ambient temperature and the acid neutralized with an appropriate amount of base to within 1 pH unit of the amino acid's isoelectric point. The solution is then cooled and the solid, water insoluble, racemized amino acid filtered, washed with water and dried under vacuum.

Alternatively, the warmed solution may be cooled to ambient temperature and passed through a column packed with 5 equivalents (with respect to the amino acid) of Dowex 50WX4-50 acidic ion exchange resin (if desired, the amide of the amino acid can be isolated simply by removing the solvent under vacuum). HCl (2.5 molar equivalents) is then added and the solution heated to reflux overnight. The solution is then cooled to ambient temperature, the Chloride ions can be removed with the use of a column of basic (OH) ion exchange resin such as Dowex 550a and the solution pumped to dryness.

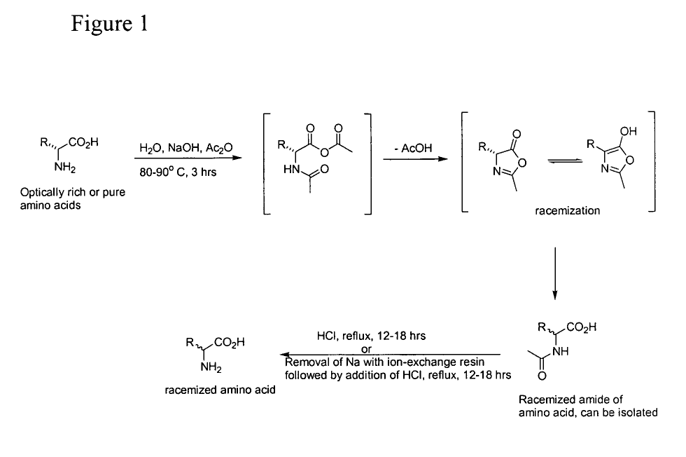

If the product desired is the N-acyl amino acid, the warmed solution is cooled to room temperature and neutralized with either an ion exchange resin (if the N-acyl amino acid has appreciable water solubility and thus freeze drying of the resultant solution) or an acid such as HCl (if the N-acyl amino acid has negligible water solubility and can thus be filtered from aqueous solutions with little loss). See FIG. 1.

### EXAMPLE 2

### Deuterium Incorporation of Amino Acids with the Use of Acetic Anhydride, Sodium Deuteroxide and Deuterium Oxide

Deuterium for hydrogen exchange of the exchangeable protons can be accomplished by either recrystallization of the amino acid or N-acyl analogs of any optical purity from D₂O or dissolution of the amino acid in D₂O followed by removal of the solvent under vacuum. The alpha-hydrogen of amino acids do not readily exchange under these conditions.

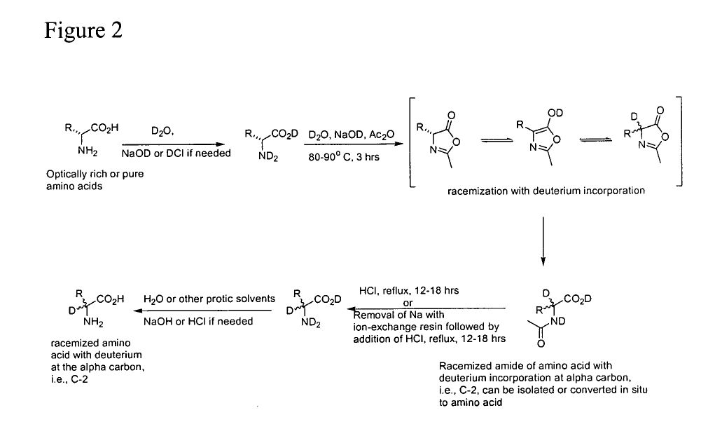

Incorporation of deuterium into the alpha-carbon of amino acids occurred using acetic anhydride and substituting D₂O, NaOD and DCl as described in example 1. Once the deuterated amino acid or N-acylamino acid is obtained, if desired, the exchangeable deuteriums can be replaced with hydrogens by either recrystallization of the amino acid from water or dissolution of the amino acid in water followed by removal of the D-rich water under vacuum. See FIG. 2.

### EXAMPLE 3

### Application of the Amino Acid Racemization to the Preparation of Alpha Deutero-p-Boronophenylalanine

D-isomer rich BPA is added to a stirring solution of NaOH in water. Once all of the BPA is dissolved, acetic anhydride is added and the solution warmed to 80-100° C. for 1 hour. Concentrated HCl is then added slowly and the reaction mixture heated to 85-100° C. for 12-18 hours. The solution filtered and cooled to 18-25° C. The pH of the solution is then adjusted to 6 (+/−0.5) with 5M NaOH solution and the solid filtered off once the solution returned to a temperature between 18 and 25° C. The white solid was washed twice with distilled water, once with acetone, and dried to constant weight to give (90% yield) racemic BPA.

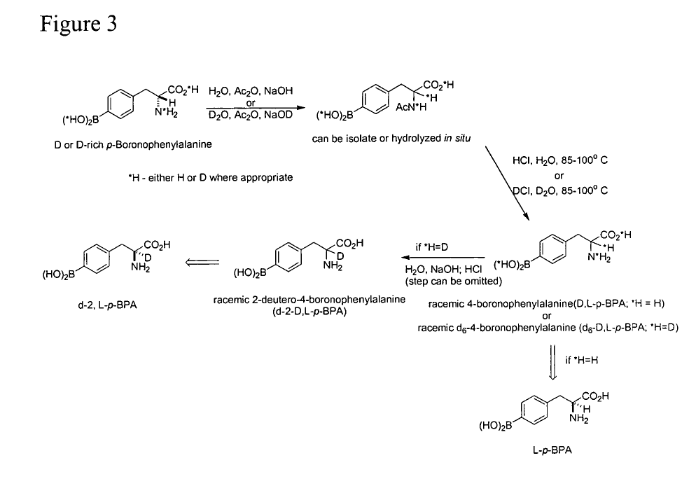

¹⁰B enriched p-boronophenylalanine (any optical purity) is dissolved in D₂O and 30% NaOD in D₂O. Once the all of the solid is dissolved, 35% DCl in D₂O is added and the solution stirred for 1 hour. The white solid is then filtered and washed with D₂O to provide the white solid d₅-p-boronophenylalanine (d₅-BPA) in which the easily exchangeable protons have been exchanged with deuterium. This solid is then dissolved in D₂O containing 30% NaOD followed by acetic anhydride and the solution heated to 80-90° C. for 2 hours. DCl (22% solution in D₂O) is added and the solution is heated to reflux overnight. The solution is cooled to 50° C. or cooler and NaOD was added until the pH of the solution is between 5 and 6. The solution is stirred overnight and the white precipitate filtered off, washed with D₂O, acetone and dried under vacuum to provide racemic d₆-BPA as a white solid. If desired, the exchangeable deuteriums could be replaced with hydrogens in almost quantitative yield by dissolving the d₆-BPA in water and a minimum amount of NaOH, followed by neutralization with concentrated HCl to provide ¹⁰B enriched alpha-deutero-p-boronophenylalanine as a white solid (deuterium incorporation >98%). See FIG. 3.

### EXAMPLE 4

### Preparation of d₅-(2,2-Dicarboxy-2-Acetamidoethyl) Benzeneboronic acid

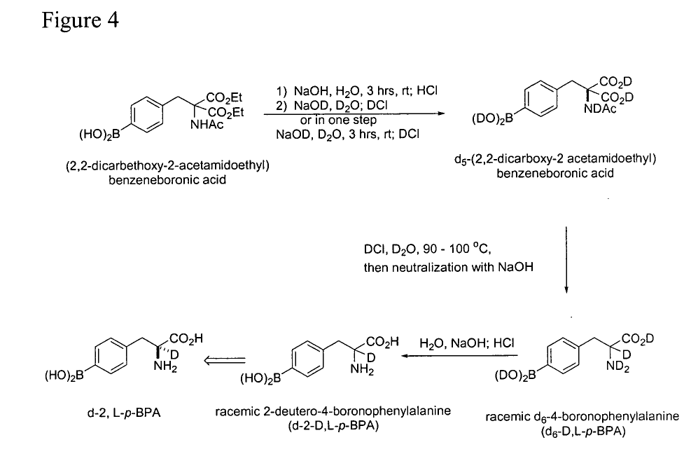

2,2-Dicarbethoxy-2-acetamidoethyl-benzeneboronic acid (400 g) was dissolved in 3 L H₂O containing 120 g NaOH and stirred for 3 hrs. The solution was then filtered through glass wool to remove any undissolved material. Concentrated HCl was then added to the filtrate and the solution stirred for 3 hours after which a white solid precipitated out of solution. The solution was then was filtered and the solid washed with water to provide 315 g (2,2-dicarboxy-2-acetamidoethyl) benzeneboronic acid as a white solid (94%). A portion of this solid (12.0 g) was then dissolved in 140 g D₂O containing 5.2 g 40% NaOD in D₂O and then precipitated by the addition of 5.5 g 35% DCl in D₂O. After stirring for 2 hrs, the solution was filtered and the solid washed with D₂O to provide, after drying, 11.5 g (94%) d₅-(2,2-dicarboxy-2-acetamidoethyl)benzeneboronic acid as a white solid (the NH, B(OH)2, and two CO₂H functionalities changed to ND, B(OD)₂ and two CO₂D, respectively, with approximately 95% deuterium incorporation). Alternatively, (2,2-Dicarbethoxy-2-acetamidoethyl)-benzeneboronic acid (10 g) was dissolved in 100 g D₂O and 7.5 g 40% NaOD in D₂O and stirred for 3 hrs. The solution was then filtered through glass wool to remove any solid impurities and 8.0 g 35% DCl in D₂O was added and the solution was stirred for 3 hrs after which a white solid precipitated out of solution. The solution was filtered and the solid was washed with D₂O to provide 8.04 g (94%) d₅-(2,2-dicarboxy-2-acetamidoethyl) benzeneboronic acid as a white solid with the deuterium content of the exchangeable protons approximately 95%. See FIG. 4.

### EXAMPLE 6

### d₆-p-boronophenylalanine (D₆-BPA) and alpha-Deutero-p-boronophenylalanine (2-D-BPA)

d₅-(2,2-dicarboxy-2-acetamidoethyl)benzeneboronic acid was dissolved in 350 g D₂O containing 16 g 35% DCl in D₂O and heated to reflux overnight. The solution was then cooled to about 50° C. and passed through sintered glass filter to remove any solid impurities. NaOD (16 g of a 40% solution in D₂O) was then added and the solution stirred for several hrs. The solid precipitate was filtered and washed with D₂O, acetone and dried to provide 11.2 g (81%) d₆-boronophenylalanine as a white solid. The exchangeable D's can be replaced with H's by dissolving the d₆-boronophenylalanine in basic water followed by neutralization and filtration to provide racemic 2-deutero-p-boronophenylalanine as a white solid with a minimal loss of material (95-99%). The deuterium content at the alpha carbon is typically >98%, dependent upon the deuterium content of the starting d₅-(2,2-dicarboxy-2-acetamidoethyl)benzeneboronic acid and D₂O used. See FIG. 4.

### EXAMPLE 7

### Preparation of ¹⁰B Enriched alpha-deutero-p-boronophenylalanine-D-Fructose Complex

¹⁰B enriched alpha-deutero-p-boronophenylalanine and D-fructose are suspended in distilled water (3 mL), followed by the addition of 0.5 M NaOH with gentle heating. The pH of this solution is typically between 8.5 and 9.0. The pH of the solution is immediately lowered (within 2-3 minutes) to between 7.3 and 7.5 by the addition of Dowex 50WX4-50 ion exchange (H⁺) resin with vigorous stirring. The solution is removed from the resin. The room temperature solution is then filtered and then freeze-dried to give ¹⁰B enriched sodium alpha-deuterated p-boronophenylalanine-D-fructose complex.

### EXAMPLE 8

### 3D ²H MRI Images of Model for Human Breast Cancer Tumors with ¹⁰B Enriched alpha-deutero-p-boronophenylalanine-D-Fructose Complex

Parametric images provide a means to quantitatively evaluate ¹⁰B enriched alpha-deutero-p-boronophenylalanine-D-Fructose perfusion in tumors. Volume distribution of ¹⁰B Enriched alpha-deutero-p-boronophenylalanine-d-Fructose is viewed inside of a tumor. Imaging is done using procedures provided in Bogin et al., “Parametric Imaging of Tumor Perfusion with Deuterium Magnetic Resonance Imaging” Microvascular Research 64, 104-115 (2002) and references cited therein. MCF7 human breast cancer cells are inoculated subcutaneously into the mammary gland of female CD1-NU athymic mice. Prior to injection of cells, a pellet of 17-estradiol is implanted under the skin of the back. Tumors are allowed to develop. For the MRI measurements, mice are anesthetized. A solution comprising ¹⁰B enriched alpha-deuterated boronophenylalanine-D-Fructose complex is slowly infused into the tail vein.

Magnetic resonance images are recorded with a spectrometer. Both ²H and ¹H images are recorded at two frequencies and detected by a double-tuned ²H/¹H surface coil system. Dynamic ²H MR images are acquired utilizing a 3D gradient echo sequence. This sequence is designed to achieve optimal signal-to-noise ratio (SNR) based on MRS T₁ measurements ²H(T₁). The sequential acquisition of ²H images begins with a preinfusion image and continued throughout infusion as described above, and for regular time intervals thereafter. The preinfusion images are recorded with a tube containing alpha-deuterated boronophenylalanine in saline attached to the tumor. This served to calibrate alpha-deuterated boronophenylalanine-D-Fructose concentrations. ¹H T₂-weighted rapid acquisition with relaxation enhancement spin-echo multislice images are used, prior to the dynamic ²H MRI, with a TE/TR at two spatial resolutions. Hennig et al., “RARE imaging: A fast imaging method for clinical MR” Magn. Reson. Med. 3, 823-833 (1986).

Image analysis is applied to the time evolution of ²H intensity in a series of coronal images. These images are reconstructed from dynamic 3D images of the whole tumor acquired before, during, and after infusion of deuterated water. Pixel-by-pixel analysis is performed utilizing a nonlinear least-squares-fitting Algorithm. For each pixel in a series of ²H images, the output of the model-based algorithm is three parametric images of perfusion rate, intravascular volume fraction, and a proportion of variability, which reflected the quality of the fitting. The effect of spatial resolution is examined by performing image analysis at descending pixel resolutions. An automated program averages the intensities in adjacent pixels in the original MR images to create images whose resolution was lower on each dimension in the plane.

### EXAMPLE 9

### Synthesis of p-boronophenylalanine-polyol or -aminopolyol Complexes

L-p-Boronophenylalanine-polyol or -aminopolyol complexes are prepared by combining 0.5 mM of alpha-deuterated or alpha-hydrogenated L-p-BPA with between 1.00 and 1.05 equivalents of complexing agent (with the exception of poorly complexing polyols and carbohydrates, in which up to 4.3 equivalents of polyol or carbohydrates are used) in 2 mL of water. One equivalent of NaOH (1 mL of 0.5 M) is then added and the slurry gently warmed if needed in order to dissolve all solids. The pH is then monitored and reduced to between 7.5 and 7.3 by the addition of Dowex50WX4-50 ion exchange resin. The clear solution is then passed through a 0.2 μm syringe filter and then freeze-dried to constant weight. ¹H and ¹³C NMR spectra are recorded on a GE 300 MHz FT-NMR spectrometer with D₂O buffered at 7.4 [Bates, R G, Bower, V E. 1956. Alkaline solutions for pH control. Anal. Chem. 28:1322-1324] as the solvent. The binding constants are calculated at three different concentrations by integration of the free L-p-BPA aromatic protons (7.73 and 7.33 ppm) compared with those of the L-p-BPA complexes (7.5 and 7.2 ppm). Spectra of the 1:1 complexes are reported.

L-p-boronophenylalanine-xylitol complex ¹H NMR δ 7.52 (d, J=7.3 Hz, ²H), 7.20 (d, J=7.3 Hz, 2H), 3.95 (dd, J=8.1, 5.1 Hz, 1H), 3.25 and 3.02 (ABq, J_{AB}=14.7 Hz, the peaks at 3.25 and 3.02 are further split into d with J=5.1 and 8.1, respectively, 2H); ¹³C NMR δ 177.0 (s), 146.0 (s), 135.8 (s), 135.1 (d, 2C), 130.9 (d, 2C), 77.8 (d), 74.6 (d), 73.4 (d), 66.0 (t), 65.3 (t), 58.8 (d), 39.0 (t).

L-p-boronophenylalanine-rhamnitol complex ¹H NMR δ 7.54 (d, J=7.3 Hz, ²H), 7.21 (d, J=7.3 Hz, ²H), 4.10 (m, 1H), 3.95 (dd, J=8.1, 5.1 Hz, 1H), 3.55-3.95 (m, 5H), 3.26 and 3.03 (ABq, J_{AB}=13.9 Hz, the peaks at 3.26 and 3.03 are split further into d with J=5.1 and 8.1, respectively, 2H), 1.24 (d, J=6.6 Hz, 3H); ¹³C NMR δ 177.0, 146.3, 136.1, 135.3 (2C), 131.0 (2C), 82.0, 78.4, 74.5, 71.8, 66.4, 58.8, 39.1, 21.1.

L-p-boronophenylalanine-sorbitol complex ¹H NMR δ 7.50 (d, J=7.3 Hz, ²H), 7.17 (d, J=7.3 Hz, ²H), 4.08 (m, 1H), 3.93 (dd, J=8.1, 5.1 Hz, 1H), 3.4-4.0 (m, 7 H), 3.25 and 3.02 (ABq, J_{AB}=14.7 Hz, the peaks at 3.25 and 3.02 split further into d with J=5.1 and 8.1, respectively, 2H); ¹³C NMR δ

L-p-boronophenylalanine-mannitol complex ¹H NMR δ 7.51 (d, J=6.6 Hz, ²H), 7.21 (d, J=6.6 Hz, ²H), 3.97 (dd, J=8.1, 5.1 Hz, 1H), 3.6-4.0 (m, 8H), 3.26 and 3.02 (ABq. J_{AB}=14.7 Hz, the peaks at 3.26 and 3.02 are split further into d with J=5.1 and 8.1, respectively, 2H); ¹³C NMR δ 177.0 (s), 146.0 (s), 135.8 (s), 135.1 (d, 2C), 131.0 (d, 2C), 78.2 (d), 74.3 (d), 73.5 (d), 71.9 (d), 66.4 (t), 65.9 (t), 58.8 (d), 39.0 (t)

L-p-boronophenylalanine-dulcitol complex ¹H NMR δ 7.50 (d, J=7.3 Hz, ²H), 7.19 (d, J=7.3 Hz, ²H), 3.95 (dd, J=8.1, 5.1 Hz, 1H), 3.90-4.02 (m, 2H) 3.50-3.78 (m, 6H), 3.27 and 3.02 (ABq, J_{AB}=14.7, the peaks at 3.27 and 3.02 are further split into d with J=5.1 and 8.1, respectively, 2H); ¹³C NMR δ 177.0(s), 146.0(s), 135.9 (s), 135.1 (d, 2C), 130.9 (d, 2C), 72.8 (d, 2C), 72.1 (d, 2C), 65.9 (t, 2C), 58.8 (d), 39.0 (t).

L-p-boronophenylalanine-aminodulcitol complex ¹H NMR δ 7.51 (d, J=7.2 Hz, 2H), 7.19 (d, J=7.2 Hz, 2H), 3.80-4.23 (m, 4H), 3.4-3.8 (m, 3H), 2.95-3.32 (m, 4H).

L-p-boronophenylalanine-aminosorbitol complex ¹H NMR δ 7.52 (d, J=7.3 Hz, 2H), 7.21 (d, J=7.3 Hz, 2H), 4.31 (v br s, 1H), 4.04 (br s, 1H), 3.91 (dd, J=8.0, 5.1 Hz, 1H), 3.55-3.85 (m, 4H), 3.24 and 3.04 (ABq, J_{AB}=14.3 Hz, the peaks at 3.24 and 3.04 are split further into d with J=5.1 and J=8.1 Hz, respectively, 2H), 2.90-3.15 (m, 2 H); ¹³C NMR δ 176.9 (s), 146.0 (s), 136.0 (s), 134.9 (d, 2C), 131.1 (d, 2C), 78.2 (d), 76.0 (d), 70.8 (d), 68.5 (d), 65.9 (t), 58.7 (d), 46.3 (t), 38.9 (t).

L-p-boronophenylalanine-maltitol complex ¹H NMR δ 7.51 (d, J=3 Hz, 2H), 7.19 (d, J=7.3 Hz, 2H), 5.11 (d, J=3.7Hz, 1H); ¹³C NMR δ 176.9(s), 146.3(s), 135.8 (d, 2C), 135.1 (d, 2C), 98.6 (d), 74.4, 75.0, 74.7, 74.6, 73.9, 72.1, 66.0, 63.0, 58.7, 38.9 (t).

L-p-boronophenylalanine-isomaltitol complex ¹H NMR δ 7.49 (d, J=7.3 Hz, 2H), 7.19 (d, J=7.3 Hz, 2H), 4.94 (d, J=3.7 Hz, 1H), 4.09 (m, 1H), 3.45-4.05 (m, 13H), 3.43 and 3.39 (ABq, J_{AB}=9.5 Hz, 2H), 3.95 (dd, J=8.8, 4.4 Hz, 1H), 3.27 and 3.01 (ABq, J_{AB}=13.9 Hz, the peaks at 3.27 and 3.01 are further split into d with J=4.4 and 8.8 Hz, respectively, 2H).

L-p-boronophenylalanine-cellobiitol complex ¹H NMR δ 7.52 (d, J=7.3 Hz, 2H), 7.19 (d, J=7.3 Hz, 2H), 4.56 (d, J=7.3 Hz, 1H), 4.34 (br s, 1H), 4.16 (dd, J=6.6, 6.6 Hz, 1H), 3.66-3.95 (m, 7H), 3.22-3.58 (m, 5H), 3.25 and 3.02 (ABq, J_{AB}=14.3 Hz, the peaks at 3.25 and 3.02 split further into d with J=5.1 and J=8.1 Hz, respectively, 2H); ¹³C NMR δ 176.9 (s), 147.0 (s), 135.8 (s), 135.1 (d, 2C), 130.9 (d, 2C), 106.3 (d), 79.2 (d), 78.4 (d), 78.3 (d), 76.2 (d), 75.8 (d), 74.9 (d), 74.5 (d), 72.3 (d), 66.1 (t), 65.7 (t), 63.4 (t), 58.7 (d), 38.9 (t).

L-p-boronophenylalanine-lactitol complex ¹H NMR δ 7.50 (d, J=7.3 Hz, 2H), 7.19 (d, J=7.3 Hz, 2H), 4.49 (d, J=7.3 Hz, 1H), 4.38 (br s, 1H), 4.17 (dd, J=6.6, 6.6 Hz, 1H), 3.95 (dd, J=8.1, 4.4 Hz, 1H), 3.4-4.0 (m, 12H), 3.27 and 3.02 (ABq, J_{AB}=14.7 Hz, the peaks at 3.27 and 3.02 are further split into d with J=4.4 and 8.1 Hz, respectively).; ¹³C NMR δ 176.8 (s), 146.2 (s), 135.8 (s), 135.0 (d, 2C), 130.8 (d, 2C), 106.8 (d), 179.0 (d), 77.5 (d), 76.2 (d), 75.2 (d), 74.8 (d), 74.5 (d), 73.4 (d), 71.1 (d), 65.8 (t), 65.6 (t), 63.5 (t), 58.6 (d), 38.8 (t).

L-p-boronophenylalanine-α-glucoheptitol complex ¹H NMR δ 7.52 (d, J=7.3 Hz, 2H), 7.19 (d, J=7.3 Hz, 2H), 4.23 (s, 1H), 3.85-4.05 (m, 2H), 3.95 (dd, J=8.8, 4.4 Hz, 1H), 3.50-3.85 (m, 6H), 3.27 and 3.02 (ABq, J_{AB}=14.7 Hz, the peaks at 3.27 and 3.02 are further split into d with J=4.4 and J=8.8 Hz, respectively, 2H).

L-p-boronophenylalanine-persietol complex ¹H NMR δ 7.51 (br d, J=6.6 Hz, 2H), 7.20 (br d, J=6.6 Hz, 2H), 3.5-4.1 (m, 9H), 3.93 (dd, J=8.8, 3.7 Hz, 1H), 3.25 and 3.02 (ABq, J_{AB}=13.9, the peaks at 3.25 and 3.02 are further split into d with J=3.7 and 8.8 Hz, respectively, 2H).

L-p-boronophenylalanine-volemitol complex ¹H NMR δ 7.50 (br d, J=6.6 Hz, 2H), 7.20 (br d, J=6.6 Hz, 2H), 3.55-4.07 (m, 9H), 3.93 (dd, J=8.0, 4.4 Hz, 1H), 3.25 and 3.02 (ABq, J_{AB}=14.6 Hz, the peaks at 3.25 and 3.02 are further split into d with J=4.4 and 8.0 Hz, respectively, 2H).

L-p-boronophenylalanine-α,α-glucooctitol complex ¹H NMR δ 7.51 (br d, J=7.3 Hz, 2H), 7.18 (br d, J=7.3 Hz, 2H), 4.24 (s, 1H), 4.05 (m, 1 H), 3.92 (dd, J=8.0, 3.7 Hz, 1H), 3.50-4.00 (m, 8H), 3.25 and 3.02 (ABq, J_{AB}=14.7, the peaks at 3.25 and 3.02 are further split into d with J=3.7 and 8.0 Hz, respectively, 2H).

### EXAMPLE 10

### Application of the Amino Acid Racemization to the Preparation of alpha ³H-Boronophenylalanine

p-Boronophenylalanine is dissolved in tritiated water-[H³]. Li³H synthesized from tritium gas as described in Than et al., J Org Chem. 1996 13;61(25):8771-8774 is added followed by acetic anhydride. The solution is heated to reflux. After allowing the solution to cool to room temperature, concentrated HCl_{(aq)} is added and the solution is heated to reflux overnight. After allowing the solution to cool to room temperature, the solution was brought to a pH of 6 by the addition of sodium hydroxide. The solvents are removed by lyophilization and the salts are removed by column using ion exchange resins to give a composition comprising a mixture of p-BPA and alpha-[H³] pBPA.

Both ³H and ¹H images are recorded at two frequencies and detected by a double-tuned ³H/¹H surface coil system. Parameters are obtained from Vogt et al., “Improved methods for ¹H-³H heteronuclear shift correlation” Magn Reson Chem. 2005;43(2):147-55 and Kubinec et al., “Applications of tritium NMR to macromolecules: a study of two nucleic acid molecules” J Biomol NMR. 1996;7(3):236-46. The sequential acquisition of ³H images begins with a preinfusion image and continued throughout infusion as described above, and for regular time intervals thereafter. Hennig et al., “RARE imaging: A fast imaging method for clinical MR” Magn. Reson. Med. 3, 823-833 (1986).

We claim:

1. A method comprising
a) providing
i) a subject comprising mammalian tissue,
ii) a compound comprising a deuterium atom, and
iii) a nuclear magnetic resonance spectrometer configured to detect said deuterium atom;
b) administering said compound to said subject; and
c) detecting said deuterium atom with said nuclear magnetic resonance spectrometer in said tissue.

2. The method of claim 1, wherein said compound comprises an alpha-deutero-amino acid.

3. The method of claim 1, wherein said compound further comprises boron.

4. The method of claim 2, wherein said alpha-deutero-amino acid is alpha-deutero-boronophenylalanine.

5. The method of claim 4, wherein said alpha-deutero-boronophenylalanine is an alpha-deutero-boronophenylalanine saccharide conjugate.

6. The method of claim 5, wherein said alpha-deutero-boronophenylalanine saccharide conjugate is selected from the group consisting of:
alpha-deutero p-boronophenylalanine-α,α-glucooctitol;
alpha-deutero p-boronophenylalanine-volemitol;
alpha-deutero p-boronophenylalanine-persietol;
alpha-deutero p-boronophenylalanine-α-glucoheptitol;
alpha-deutero p-boronophenylalanine-lactitol;
alpha-deutero p-boronophenylalanine-cellobiitol;
alpha-deutero p-boronophenylalanine-isomaltitol;
alpha-deutero p-boronophenylalanine-maltitol;
alpha-deutero p-boronophenylalanine-aminosorbitol;
alpha-deutero p-boronophenylalanine-aminodulcitol;
alpha-deutero p-boronophenylalanine-dulcitol;
alpha-deutero p-boronophenylalanine-mannitol;
alpha-deutero p-boronophenylalanine-sorbitol;
alpha-deutero p-boronophenylalanine-rhamnitol; and
alpha-deutero p-boronophenylalanine-xylitol.

7. The method of claim 1, further comprising performing boron neuron capture therapy by exposing said subject to neutron irradiation.

8. A purified isotopic composition of a compound having the following structure:

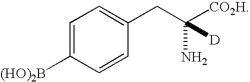

9. A composition comprising a boronophenylalanine saccharide conjugate.

10. The composition of claim 9, wherein said boronophenylalanine saccharide conjugate is selected from the group consisting of:
p-boronophenylalanine-α,α-glucooctitol;
p-boronophenylalanine-volemitol;
p-boronophenylalanine-persietol;
p-boronophenylalanine-α-glucoheptitol;
p-boronophenylalanine-lactitol;
p-boronophenylalanine-cellobiitol;
p-boronophenylalanine-isomaltitol;
p-boronophenylalanine-maltitol;
p-boronophenylalanine-aminosorbitol;
p-boronophenylalanine-aminodulcitol;
p-boronophenylalanine-dulcitol;
p-boronophenylalanine-mannitol;
p-boronophenylalanine-sorbitol;
p-boronophenylalanine-rhamnitol; and
p-boronophenylalanine-xylitol.

11. The composition of claim 9, wherein said boronophenylalanine saccharide conjugate is an alpha-deuterated boronophenylalanine.

12. The composition of claim 11 wherein said alpha-deuterated boronophenylalanine is selected from the group consisting of:
alpha-deuterated p-boronophenylalanine-α,α-glucooctitol;
alpha-deuterated p-boronophenylalanine-volemitol;
alpha-deuterated p-boronophenylalanine-persietol
alpha-deuterated p-boronophenylalanine-α-glucoheptitol;
alpha-deuterated p-boronophenylalanine-lactitol;
alpha-deuterated p-boronophenylalanine-cellobiitol;
alpha-deuterated p-boronophenylalanine-isomaltitol;
alpha-deuterated p-boronophenylalanine-maltitol;
alpha-deuterated p-boronophenylalanine-aminosorbitol;
alpha-deuterated p-boronophenylalanine-aminodulcitol;
alpha-deuterated p-boronophenylalanine-dulcitol;
alpha-deuterated p-boronophenylalanine-mannitol;
alpha-deuterated p-boronophenylalanine-sorbitol;
alpha-deuterated p-boronophenylalanine-rhamnitol; and
alpha-deuterated p-boronophenylalanine-xylitol.

13. A method of making alpha-deuterated amino acids comprising:
a) providing
i) a substituted or unsubstituted amino acid,
ii) a carboxylic acid anhydride,
iii) a solution of deuterium oxide, and
iv) a base,
b) mixing said amino acid, said solution of deuterium oxide, and said base under conditions providing a basic solution; and
c) mixing said basic solution with said carboxylic acid anhydride under conditions such that said amino acid is racemized.

14. The method of claim 13 wherein said amino acid is boronophenylalanine.

15. The method of claim 13 wherein said carboxylic acid anhydride is acetic anhydride.

16. The method of claim 13 wherein said base is selected from the group consisting of sodium deuteroxide and sodium deuteride.

17. The method of claim 13 wherein said base is presented in greater than one molar equivalent compared to the amino acid.

18. The method of claim 13, wherein said base is presented in greater than one and one-half molar equivalents compared to the amino acid.

19. The method of claim 13, wherein said base is presented in two or greater than two molar equivalent compared to the amino acid.

20. A method of racemizing amino acids comprising
a) providing
i) an amino acid or N-acylated amino acid,
ii) a carboxylic acid anhydride,
iii) a solution of water, and
iv) a base,
b) mixing said amino acid or N-acylated amino acid, said solution of water, and said base under conditions providing a basic solution; and
c) mixing said basic solution with said carboxylic acid anhydride under conditions such that said amino acid or N-acylated amino acid is racemized.

21. The method of claim 20, wherein said N-acylated amino acid is acetyl boronophenylalanine.

22. The method of claim 20, wherein said carboxylic acid anhydride is acetic anhydride.

23. The method of claim 20, wherein said base is sodium hydroxide.
